(19) United States
(12) Patent Application Publication
(10) Pub. No.: US 2008/0031935 A1
(43) Pub. Date: Feb. 7, 2008

(54) Cholinesterase Inhibitors In Liposomes And Their Production And Use

(76) Inventors: Angelika Bodenteich, Wien (AT); Josef Bockmann, Modling (AT); Werner Frantsits, Wien (AT); Eberhard Pirich, Wien (AT); Andreas Wagner, Baden (AT); Karola Vorauer-Uhl, Wien (AT)

Correspondence Address: POPOVICH, WILES & O'CONNELL, PA;650 THIRD AVENUE SOUTH, SUITE 600, MINNEAPOLIS, MN, 55402, US

(21) Appl. No.: 11/578,191

(22) PCT Filed: Apr. 21, 2005
(86) PCT No.: PCT/AT05/00138

§ 371 Date: Oct. 10, 2006

(30) Foreign Application Priority Data
Apr. 22, 2004 (AT) A 696/2004

(51) Int. Cl. A61K 9/127; A61K 31/27; A61K 31/407; A61K 31/435; A61P 25/00; A61K 31/4353; A61K 31/55; A61K 31/661

(52) U.S. Cl. 424/450; 514/136; 514/143; 514/295; 514/297; 514/319; 514/411; 514/490

(57) ABSTRACT

The invention relates to a pharmaceutical composition based on an active ingredient that is enclosed in liposomes for topical, transdermal application. The interior of said liposomes comprises an acidic, aqueous medium containing at least one cholinesterase inhibitor, preferably from the group containing donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, or an enantiomer or derivative of at least one of said compounds. In addition, the invention relates to a method for producing said composition, optionally in a sterile form and also to the use of the liposomes charged with the active ingredient in various galenic formulations for topical, transdermal application with a depot effect in the epidermis, for the prophylaxis and/or treatment of cutaneous neuropathic pain or the loss of cutaneous sensory function as a result of neuropathy.

### TECHNICAL FIELD

This invention concerns pharmaceutical compositions based on cholinesterase inhibitors in liposomes, the preparation of such compositions and their possibilities for use in therapy.

### BACKGROUND OF INVENTION

Central cholinesterase inhibitors are used for pharmacotherapy of mild and moderate Alzheimer's disease in order to partially restore the diminished function of the cholinergic conduction pathway system in the brain that is produced in this syndrome. Recent research showed that many cholinesterase inhibitors not only block acetyl- and butyrylcholinesterases by various mechanisms, but also have direct effects on neuronal nicotinic acetylcholine receptors. These receptors, whose natural ligand is acetylcholine, are found not only on cholinergic nerves, but also in serotonergic and glutamatergic nerve systems, and they control the release of the relevant neurotransmitter there.

This effect arises with quite different concentrations of the relevant agent, takes place at different binding sites of the receptor, and can result in a blockage or an enhancement of the effect of acetylcholine on the receptor, in some cases even in dependence on the concentration of the cholinesterase inhibitor itself and/or on the acetylcholine concentration that exists at the same time.

A prototypical role is ascribed to galantamine, which has been especially thoroughly investigated in this regard, since at the concentrations that bring about a therapeutically active cholinesterase inhibition, it is an allosterically modulating ligand at a binding site distinct from the acetylcholine binding site (Samochocki et al., Acta Neurol Scand Suppl. 2000; 176: 68-73; Dalas-Bailador et al., Mol Pharmacol. 2003; 64(5): 1217-26). Through this, the effect of the acetylcholine concentration that is increased through the cholinesterase inhibition of the galantamine becomes additionally potentiated. Tacrine evidently likewise binds to this and to another binding site of the receptor (Svennson and Nordberg, Neuroreport 1996, 7(13): 2201-5). Comparable evidence also exists for physostigmine (Onkojo et al., Eur J Biochem 1991, 200(3): 671-7) and for donepezil. A noncholinergic antiapoptotic effect was also reported for galantamine (Arroyo et al., Rev Neurol. 2002; 34(11): 1057-65), as well as an effect corresponding to that of nerve growth factor (NGF) (Capsoni et al., Proc Natl Acad Sci USA 2002; 99(19): 12432-7).

Although these studies were carried out in reference to the treatment of Alzheimer's disease with its specific cholinergic deficit and the related neurodegeneration, they are also very important for degenerative and painful diseases of the peripheral nervous system, especially the sensory nerves. On the one hand, the antinociceptive effect of nicotine and other substances that have an antagonistic effect on nicotinic receptors have been known for quite a long time. On the other hand, in the early studies of diabetic sensory neuropathy and in cases of acute nerve lesions, the concentration of NGF in the affected regions of the skin was highly reduced. NGF, or a neurotropic activity corresponding to it, which is mediated directly or via the cholinergic system, which is closely linked to NGF, could reproduce the sensory function here. However, studies in this regard were less successful, presumably because sufficiently high local NGF concentrations could not be achieved without systemic side effects (Anand, Prog Brain Res. 2004; 146: 477-92). Moreover, nicotinic agonists have shown adverse effects in their therapeutic usefulness in this connection because of side effects like high blood pressure and neuromuscular paralysis. Peripherally acting cholinesterase inhibitors proved to be unsuitable for analgesic therapy in humans, principally also because of the side effect problems (Ghelardini et al., Presynaptic Auto and Heteroreceptors in the Cholinergic Regulation of Pain. In: Trends in Receptor Research, Elsevier Science Publishers B.V., 1992).

Transdermal formulations of cholinesterase inhibitors based on patches that contain the active agent dissolved or distributed in a dermal penetration enhancer are well known. The following may be mentioned as examples of such passive transdermal systems: EP 0 376 067, EP 0 377 147, EP 0 667 774, EP 0 599 952 and EP 0 517 840 for physostigmine; WO 99/34782 for rivastigmine; EP 0 680 325 for galantamine, and WO 01/32115 for huperzine. Moriearty et al. (Methods Find Exp Clin Pharmacol 1993; 15(6): 407-12) describes such systems for metrifonate or its hydrolysis product dichlorvos as a cholinesterase inhibitor, and for heptylphysostigmine. Two other synthetic derivatives of physostigmine, thiacymserine and thiatolserine, were expressly described by their developer as especially suitable for transdermal use.

These transdermal systems were all intended for uses that require systemic administration of the relevant active agents. The transdermal route in these cases is selected because of the desire to release the active agents into the bloodstream slowly and uniformly and/or to avoid the “first pass” degradation in the liver that occurs with oral ingestion, so that therapeutically optimum plasma levels continue to exist for a long time. The systemic effect is achieved by the active agent penetrating the skin through passive diffusion and being carried into the bloodstream by subdermal capillary vessels. The active agent can also be temporarily deposited or bound in subcutaneous fatty tissue in order to be slowly washed from this tissue into the circulation. Only slight retention of the active agent intentionally arises in the skin itself. Said systems therefore are not suitable for treatment of neuropathic pain or a reduction of the dermal sensory function due to neurodegeneration.

Liposomes are known as means for controlled release of pharmaceutical agents (for example, see the overview in Ullrich, Biosci Rep. 2002; 22(2): 129-50), especially for use in special transdermal “patch” systems (for example, those published in WO 87/01938 and U.S. Pat. No. 5,718,914) and in gels (U.S. Pat. No. 5,064,655). The formulation of local anesthetics in topically applied liposomes is also known to the specialist; for example, U.S. Pat. No. 4,937,078 describes liposomes that contain conventional sodium channel blockers like tetracaine, lidocaine and so forth.

A liposomal formulation of the cholinesterase inhibitor neostigmine for use as an analgesic has been described (Grant et al., Acta Anaesthesiol Scand. 2002; 46(1): 90-4), but it was administered intrathecally (i.e., into the connective tissue), so that the observed analgesic effect was a central effect.

### BRIEF DESCRIPTION OF INVENTION

Thus, the task in accordance with the invention is to find a way to administer cholinesterase inhibitors that have known effect on neuronal nicotinic receptors and/or NGF-like neurotropic activity in order to reduce or avoid both the known disadvantages of patch applications (for example, the need for a surface that is as flat as possible for application of the patch; possible skin irritations; active agent concentration in patch must be very high; penetration enhancers can cause skin damage) and the disadvantages of invasive administration methods and other systemic modes of use, in particular the undesirable systemic side effects that are linked to the necessary high dosage.

Therefore, it is a goal of this invention to make available a pharmaceutical composition containing cholinesterase inhibitors that enables an overall low, but at the same time sufficiently high, local dosage of such active agents in the region of sensory nerve endings of the skin while at the same time largely avoiding systemic ingestion of them.

Another goal of the invention is to make available a composition that can be administered anywhere on the body, especially on “uneven” sites that are not suitable for the use of a patch, for example on the feet in cases of diabetic neuropathy, or on the face in cases of trigeminal neuralgia.

Another goal of the invention is to make available a composition that does not lead to maceration phenomena on the skin, which is particularly essential in cases of skin that has already been damaged and/or is fragile, for example, in diabetics.

Another goal of the invention is to make available a composition that can be prepared as a sterile product and thus can be applied as a therapeutic agent, for example, in cases of herpes zoster, even in the blister stage or in the healing phase.

Finally, a goal of the invention is also to make available a composition that produces an active agent depot in the skin, from which a substance is continuously released, so that better bioavailability and longer half-lives are also achieved by comparison with systemic administration.

In accordance with the invention, these goals are achieved by making available a liposomal system for topical administration of cholinesterase inhibitors.

Surprisingly, it turned out that by enclosing cholinesterase inhibitors in liposomes of a certain composition and size and then formulating these liposomes in suitable galenical systems for transdermal administration, said goals can be attained. The scalable method for active agent encapsulation disclosed in WO 02/36257 for the preparation and loading of liposomes with active agents proved to be especially advantageous because of its high efficiency, while at the same time having extremely mild process conditions. However, other methods for preparation and loading of liposomes from the prior art can also be used.

### DETAILED DESCRIPTION OF INVENTION

The loading of liposomes with active agents can be divided into two main categories: loading the membranes and loading the intraliposomal aqueous phase. Since galantamine base is soluble in ethanol, incorporation of the active agent molecules into the liposomal membrane was attempted initially, but this was not successful. The more galantamine there was that was not enclosed in the membrane and instead was removed by filtration, the more there was that was released back by the membrane. The amount of membrane-bound and non-bound galantamine was approximately the same. For this reason, loading the intraliposomal aqueous phase with galantamine was then tried. This procedure can be carried out in two different ways: by active and passive loading. Based on experiments with passive loading of liposomes with galantamine HBr/base in PBS solution, it soon turned out that stable galantamine-liposome suspensions could not be made in this way. As in the previous membrane experiments, the active agent also diffused out from the aqueous environment of the liposomes as soon as the non-enclosed active agent was removed from the surrounding medium.

For this reason, it was necessary to examine the active agent galantamine a little more closely. It was found that galantamine has similar chemical properties to doxorubicin and can be enclosed in liposomes by pH gradient-controlled loading. For this type of active loading, the most important characteristic is the liposome membrane/liposome medium distribution coefficient. It was found that the octanol/buffer distribution coefficient gives a good indication of the transmembrane diffusion of a substance and therefore is relevant for loading with active agent or for the release profile. In addition, the active agent must contain protonatable amino groups, so that the active agent is hydrophilic at low pH and lipophilic at neutral or alkaline pH.

On the basis of this theoretical model, liposomes with various lipid compositions (preferably with long-chain phospholipids and low cholesterol concentrations) were prepared in a suitable loading buffer, chiefly in a citric acid/sodium carbonate buffer. After the liposomes had been prepared at low pH, the surrounding medium was made alkaline and in this way a pH gradient was generated. After adding galantamine to the alkalinized medium, the active agent, due to this pH gradient, migrated into the liposomes, became protonated there, and remained stable in the liposomes.

When this technique is used, the extent of loading or the loading capacity is determined first of all by the ratio of the pH values within and without the liposomes. In the experiments that were conducted, values similar to those known from the literature for actively loaded liposomes are achieved with active agent/lipid ratio in the range of 200-400 nmol active agent per μmol lipid. An increase of the active agent concentration in the loading medium did not lead to an increase of the loading capacity.

The active loading described above is a three-step operation consisting of vesicle formation, active agent addition, and alkalinization. For this reason, another goal of the invention was to establish a one-step preparation method that could be implemented using the crossflow model disclosed in WO 02/36257. For this purpose, galantamine was dissolved in citric acid solution, enclosed in liposomes by means of a crossflow injection technique, and the residual citric acid solution was alkalinized immediately thereafter with a dilution buffer (citric acid/sodium carbonate, pH 9.0-9.5).

In the following examples it is shown, among other things, that the quality of the active agent-loaded liposomes can be improved merely by varying, especially by reducing, the cholesterol content in the vesicle membrane, especially with regard to the skin penetration capacity. Moreover, stability tests for the products from the three-step and the one-step methods confirm that the product stability and quality remained unchanged in both products, even after six months of storage.

Where necessary or desirable, the loading capacity can be increased further by increasing the average liposome size of about 150-200 nm (as in most of the experiments described herein) to 300-500 nm. In addition, the efficiency of the method, i.e., the amount of liposomally enclosed active agent per nmol of suspension, can also be further improved by increasing the lipid concentration either during the preparation or in the subsequent filtration of the vesicles.

### BRIEF DESCRIPTION OF THE FIGURES

FIG. 1 shows a schematic drawing of an apparatus for preparation of liposomes.

FIG. 2 shows HPLC results of galantamine inclusion experiments in liposomes. The solid bars represent galantamine in the retentate (i.e., liposomal galantamine), the shaded bars represent galantamine in the filtrate (unenclosed galantamine) and the unshaded bars represent the total amount of galantamine; Y-axis: galantamine in μg/mL.

FIG. 3 shows HPLC results of galantamine inclusion experiments in liposomes. The solid bars represent galantamine in the retentate (i.e., liposomal galantamine), the shaded bars represent galantamine in the filtrate (unenclosed galantamine) and the unshaded bars represent the total amount of galantamine; first three bars: positively charged liposomes with stearylamine; last three bars: negatively charged liposomes with E-PG; 3A: galantamine HBr; 3B: galantamine base.

FIG. 4 shows HPLC results of galantamine inclusion experiments in liposomes. The solid bars represent galantamine in the retentate (i.e., liposomal galantamine), the shaded bars represent galantamine in the filtrate (unenclosed galantamine) and the unshaded bars represent the total amount of galantamine.

FIG. 5 shows the results of a stability test with actively loaded galantamine liposomes at various aqueous phase pH values; Y-axis: active agent concentration in nmol active agent per μmol lipids; X-axis: time in weeks since manufacture of preparations.

FIG. 6 shows HPLC data on the loading of preformed liposomes with galantamine as a function of temperature and loading time. Data in percent of supplied galantamine concentration.

FIG. 7 shows HPLC data from two preparation experiments for actively loaded liposomes in the presence of an excess of galantamine. The solid bars represent the amount of unenclosed galantamine, the shaded bars represent the amount of liposomally enclosed galantamine. The light lines between the triangular symbols (pertinent values: right hand Y-axis) indicate the stable lipid/active agent ratio.

FIG. 8 shows stability data of a liposome preparation in which the liposomes were actively loaded with galantamine in a one-step process. Y-axis: active agent concentration in nmol active agent per μmol lipid; X-axis: time in weeks since preparations were produced.

FIG. 9 shows stability data of actively loaded galantamine liposomes: A) prepared in three-step process; B) prepared in one-step process.

FIG. 10 shows galantamine inclusion rates and stability of actively loaded DMPC liposomes.

FIG. 11 shows the galantamine uptake in DPPC liposomes as a function of the cholesterol content.

FIG. 12 shows a stability test of galantamine liposomes prepared by the ammonium sulfate gradient method. F1, F2 and F3 indicate filter samples; R stands for retentate.

FIG. 13 shows the stability of galantamine liposomes with lipids of different chain lengths. (A: C16 and B: C14) in hydrogel formulations.

FIG. 14 shows the results of in vitro skin penetration studies with liposomal galantamine preparations having different lipid compositions. Y-axis: ng galantamine absolute in the relevant sample; X-axis: variations of lipid composition.

FIG. 15 shows the results of in vitro skin penetration studies with liposomal galantamine preparations after repeated application.

FIG. 16 shows the results of in vitro skin penetration studies with liposomal galantamine preparations as a function of the amount of the sample and the penetration time.

FIG. 17 shows the results of in vitro skin penetration studies with liposomal galantamine preparations as a function of the hydrogel concentration.

FIG. 18 shows the results of in vitro skin penetration studies with galantamine preparations in the form of microemulsions.

FIG. 19 shows the results of in vitro skin penetration studies with hydrogel preparations based on galantamine in free form compared to liposomally enclosed galantamine.

FIG. 20 shows the results of in vitro skin penetration studies with liposomal galantamine preparations in the form of a suspension or in the form of a gel.

FIG. 21 shows the results of in vitro skin penetration studies with liposomal galantamine preparations in various skin samples.

To better illustrate the invention, it is explained further by means of the following examples. The experiments were carried out using galantamine as active agent, either in the form of the free base or as HBr salt. However, the specialist can reasonably see that, within the scope of this invention, galantamine can also be used in the form of its enantiomers and all of its pharmacologically acceptable salts. In the same way, chemical derivatives of galantamine and its enantiomers, for instance, the molecules claimed in WO 96/12692, WO 97/40049 and WO 01/74820, insofar as these are cholinesterase inhibitors and/or nicotinic receptor modulators and/or develop neurotropic NGF-like effects, are included within the scope of this invention, where the composition and size of the liposomes can be adjusted to the physical chemical properties of these molecules.

In the same sense, it is clear to specialists that substances other than galantamine and its salts and derivatives, to the extent that such substances are cholinesterase inhibitors and/or nicotinic receptor modulators and/or develop neurotropic NGF-like effects, likewise fall within the scope of this invention. Besides all of the substances listed in the accompanying description of the prior art, such substances also in particular include the molecules mentioned in WO 02/059074, as well as the derivatives of donepezil indicated in WO 01/78728 and WO 01/98271, in particular its fluorine derivative, ER-127528.

### EXAMPLE 1

### Preparation and Stability Testing of Galantamine Liposomes

Synthetic dipalmitoylphosphatidylcholine (DPPC, Genzyme, Switzerland) and cholesterol (Solvay, Netherlands) were used to prepare vesicles. Some experiments were carried out either with hen's egg phosphatidylglycerol (E-PG; Lipoid Co., Germany) or with stearylamine (Sigma, USA), in order to introduce positive or negative charges into the liposomal membrane. Galantamine (Sanochemia AG, Austria) was used as the free base or as HBr salt in the inclusion experiments. PBS (phosphate buffered saline) or citric acid in combination with sodium carbonate were used as buffer solutions.

The liposomes were preferably prepared by means of the shear-free crossflow injection technique in accordance with WO 02/36257. This technique is highly reproducible and enables the inclusion of any active agents into liposomes. This continuous, one-step method allows unilamellar liposomes with a lipid double layer membrane (“bilayer”) with definite, preselectable average size and size distribution to be produced stably by varying the process conditions, especially the injection pressure for the lipid phase. Moreover, it can be carried out under decidedly mild process conditions and it enables the use of potentially harmful solvents and especially the use of shear forces for vesicle formation to be eliminated completely. Other advantages of this method are described in detail in WO 02/36257.

Moreover, it is possible with this method to prepare all of the reagents in a sterile or germ-free form and to conduct the liposome preparation and loading under aseptic conditions, so that a sterile or germ-free product in the form of active agent-loaded liposomes results.

Detection of enclosed galantamine was carried out by rp-HPLC (reverse phase high performance liquid chromatography), after ultrafiltration and/or difiltration in a stirred cell (Amicon, USA) or after gel filtration through Sephadex G25 columns (Pharmacia, Germany). The inhouse rp-HPLC technique allows quantitative determination of the membrane components cholesterol and the active agent galantamine in a single pass. The liposome size and size distribution were determined by photon correlation spectroscopy (PCS).

Liposome Preparation:

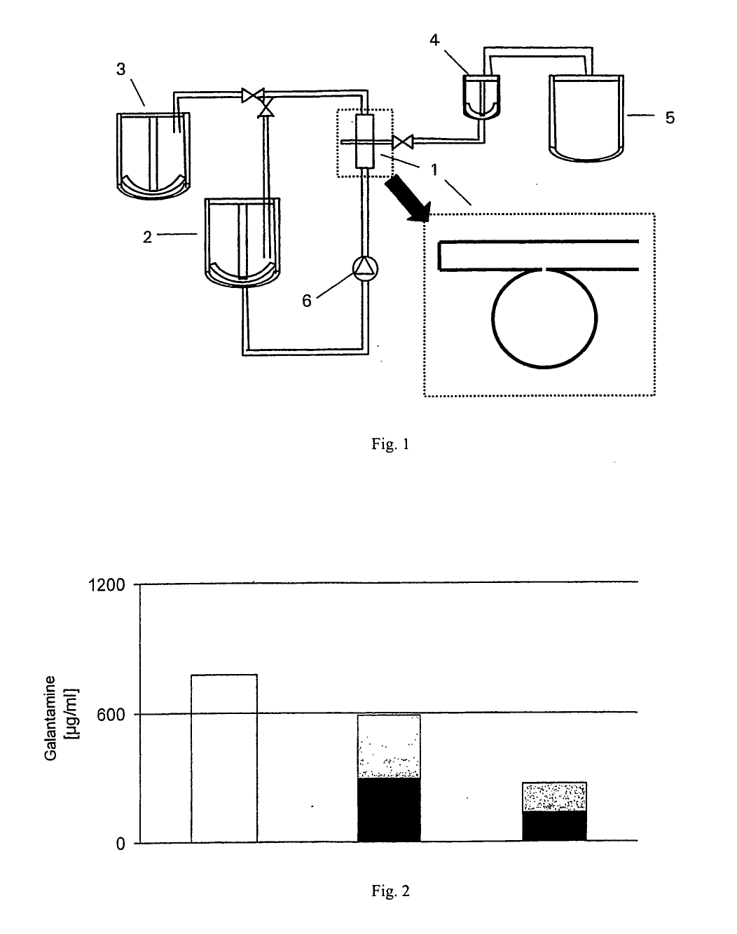

The liposomes are preferably prepared by the crossflow technique. As shown in FIG. 1, the device for liposome preparation consists of a crossflow injection module 1, containers for the polar phase (injection buffer 2 and dilution buffer 3), a container for the ethanol/lipid solution 4 and a nitrogen compressor 5. The injection orifice in the crossflow module has a diameter of about 250 μm. The lipid mixture is preferably dissolved, while stirring, in 96% ethanol at a temperature in the range of 25-60° C., according to the choice of lipid or lipid composition, for example, at a temperature of 50-55° C. in the case of DPPC liposomes. In addition, the buffer solutions are preferably heated to the same temperature, for example, 55° C. While the polar phase is pumped through the crossflow module by means of a pump 6, for example, a peristaltic pump, the ethanol/lipid solution is injected into the polar phase at the desired preset temperature at the same time.

Variant I:

DPPC, cholesterol and stearylamine (mol ratio 7:2:1) are dissolved together with galantamine in 96% ethanol and injected into PBS buffer. After the spontaneous formation of liposomes they are filtered and both the retentate and the filtrate are analyzed by rp-HPLC. As can be seen from FIG. 2, galantamine cannot be stably integrated into the liposomes in this way. The filtrate (unenclosed galantamine) and retentate (liposomally enclosed galantamine) show the same active agent concentrations.

In FIG. 2:

First bar: total amount of supplied galantamine;

Second and third bars: active agent distribution in filtrate and retentate after the first (second bar) and after an additional (third bar) difiltration.

Variant II:

Once again, the lipids were dissolved in ethanol. For experiments with negatively charged liposomes, stearylamine was replaced by hen's egg phosphatidylglycerol (E-PG). The ethanol/lipid solution was injected either into a solution of PBS/galantamine base or into a PBS/galantamine HBr solution.

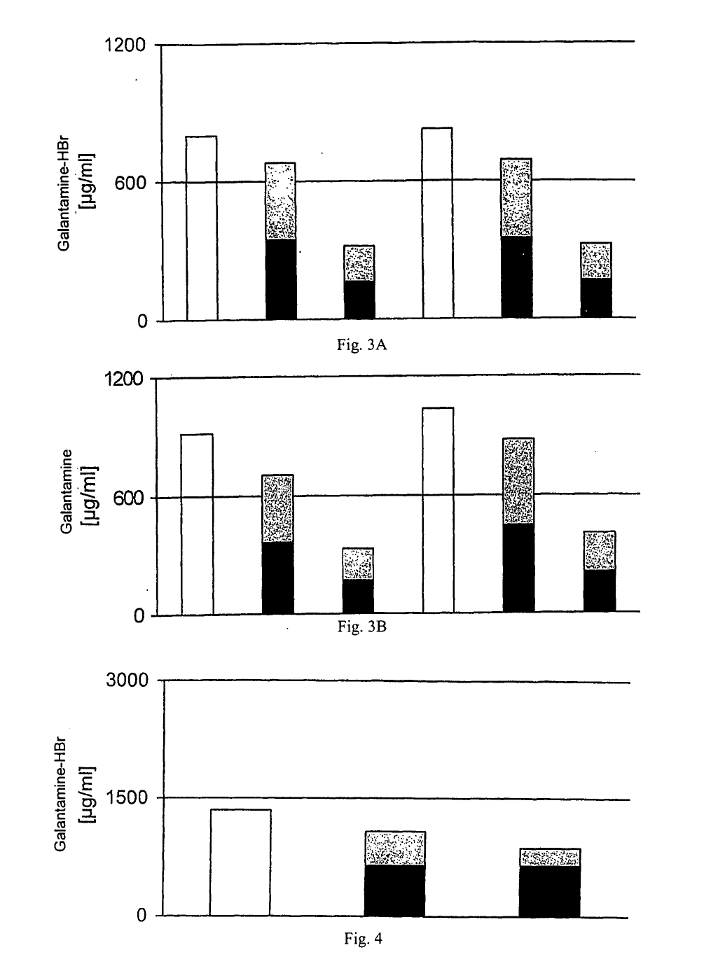

As can be seen from FIGS. 3A and 3B, galantamine could not be satisfactorily enclosed in the liposomes by this method, either. After filtration, the same amounts of galantamine were found in the filtrate and retentate.

In FIGS. 3A and 3B:

First and fourth bars: total amount of supplied galantamine in each case;

Second and third or fifth and sixth bars: active agent distribution in filtrate and retentate after the first (second and fifth bars) or after another (third and sixth bars) difiltration.

Variant III:

External loading of liposomes with galantamine by means of a pH gradient. The lipids (DPPC:cholesterol mol ratio=55:45) were dissolved in ethanol and this solution was injected into 300 mM citric acid, pH 3.5-4.5. After spontaneous vesicle formation, galantamine HBr was added and the solution was made alkaline with 500 mM sodium carbonate. In this way, a pH gradient is formed between the inside and outside of the lipid vesicles (liposomes). Because of hydrolysis problems that occur, pH values under 2.5 are less suitable, and in the same way, pH values greater than 5.5 are not preferred, because the pH gradients become increasingly less steep.

As can be seen from FIG. 4, the amount of galantamine in the filtrate is considerably less than in the retentate, which confirms that a large part of the galantamine clearly migrates along this pH gradient into the liposomes, becomes protonated there, and remains in the acid environment within the liposomes.

In FIG. 4:

First bar: total amount of supplied galantamine;

Second and third bars: active agent distribution in filtrate and retentate after the first (second bar) or after another (third bar) difiltration.

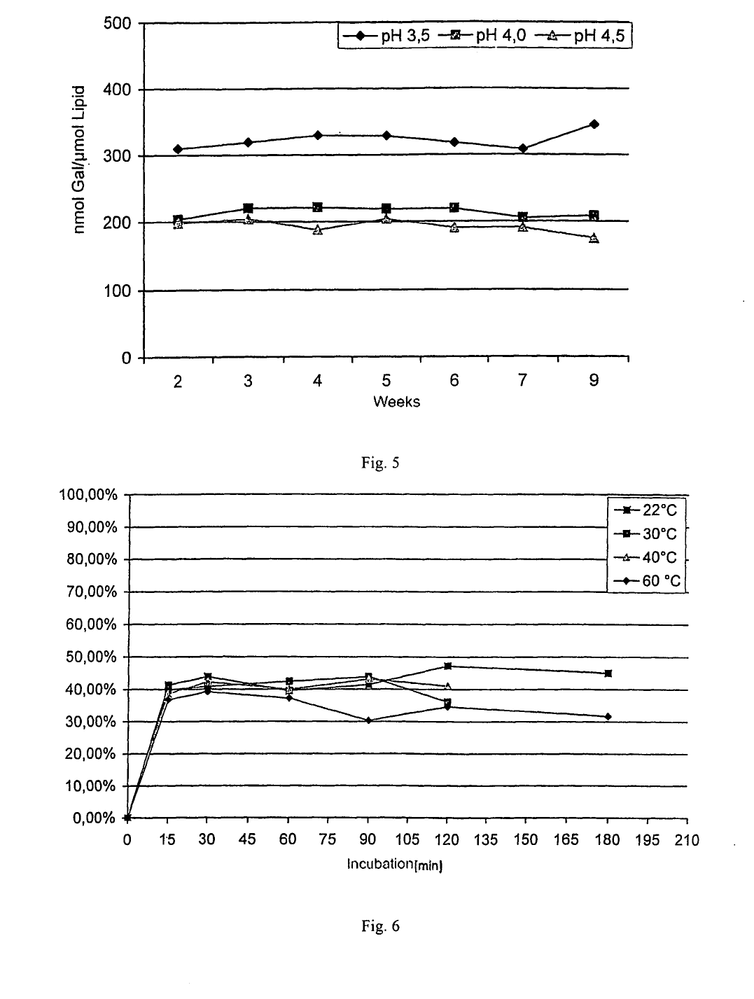

This product was divided into several aliquots and tested for stability. FIG. 5 shows the product stability over a period of nine weeks.

To determine the kinetics of the active loading of liposomes with galantamine, the time of the loading operation was investigated and the optimum loading temperature was determined. It can be seen from FIG. 6 that the total amount of galantamine migrates into the liposomes within about 15 minutes and that an incubation temperature in the range of room temperature (18-22° C.) is good both for active agent uptake and for active agent retention within the liposomes. The differences measured for a range of 22-40° C., however, are small and in any case do not play a significant role. The negative effect of higher incubation temperatures appears first of all to affect the stability of the loaded liposomes, where at a temperature of 60° C., the active agent loss after 3 h incubation already is in a range of about 20-25% (FIG. 6).

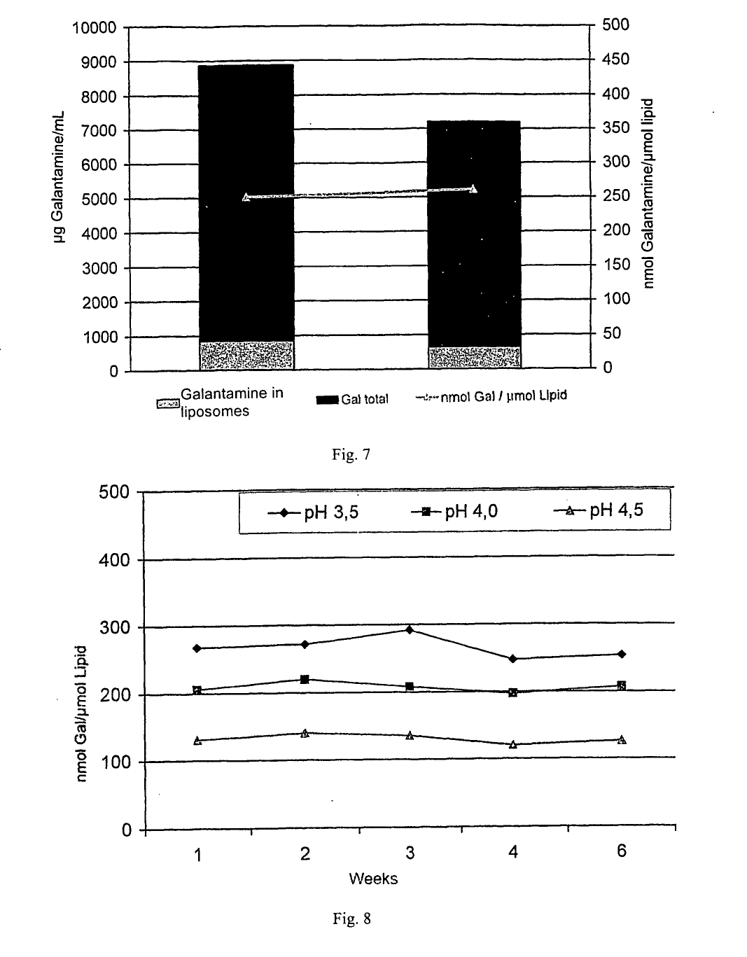

In order to achieve an increase of the amount of liposomally enclosed galantamine, a solution containing 8-10 mg galantamine per mL solution was prepared. As can be seen from FIG. 7, however, an excess of galantamine (solid bars; left hand Y-axis) cannot improve the active agent/lipid ratio, which in this experiment remains constant at about 200-300 mol galantamine per μmol lipid (light line between the triangular symbols; right-hand Y-axis). The effective loading amount in the case of active external loading over a pH gradient therefore appears to be dependent first of all on the gradient and less on the active agent concentration that is present.

Variant IV:

After the initial evaluation of the results given above, a one-step preparation process was developed. The lipids (DPPC:cholesterol=55:45 mol %) were dissolved in ethanol and the solution was injected into a galantamine HBr/citric acid solution (pH 3.5-4.5), whereupon a sodium carbonate/citric acid buffer solution (pH 9.0-9.5) was added immediately after spontaneous vesicle formation, for purposes of dilution and alkalinization of the reaction mixture, i.e., the resulting liposomal suspension. By producing this pH gradient, galantamine is not only taken up into the liposomes in one step, but it also remains there stably. The amount of such liposomally absorbed galantamine lay in a range of at least 100 nmol galantamine per μmol lipid, depending on the pH value or the pH gradient, preferably in a range of at least 150-400, for example, frequently in a range of 250-350 at a pH of 3.5 (FIGS. 8-11). This product remained stable for an observation period of six weeks (see FIG. 8).

### EXAMPLE 2

### Preparation and Comparison of Galantamine Preparations in the Form of Liposomes or Microemulsions with Variable Lipid Composition

Synthetic dipalmitoylphosphatidylcholine (DPPC, Genzyme, Switzerland), dimyristoylphosphatidylcholine (DMPC, Genzyme, Switzerland) and cholesterol (Solvay, Netherlands) were used as lipids in this example. Galantamine (Sanochemia AG, Austria) was used as the HBr salt for the liposomal inclusion studies. Citric acid/sodium carbonate was used as buffer solution.

The ammonium sulfate gradient method was employed as a second possibility for active loading. An ammonium sulfate solution and a glucose solution were used as aqueous phases for vesicle preparation. Once again, the crossflow technique was used. After removing unenclosed galantamine by gel filtration, both the active agent and lipid contents were determined by rp-HPLC. The liposome size and size distribution were again determined by photon correlation spectroscopy (PCS).

In addition to liposomes, preparations were also prepared in the form of microemulsions by vigorous mixing with stepwise heating using several heat cycles (heating to 80° C.). Isopropyl myristate (IPM) was used as oil phase. Tween and Span 20 were used as emulsifier and coemulsifier.

Liposome Design and Stability:

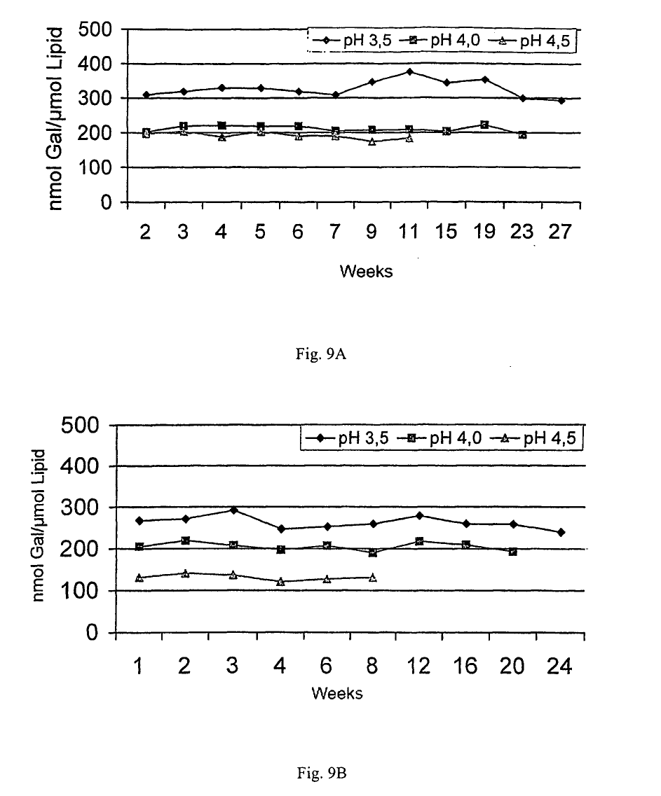

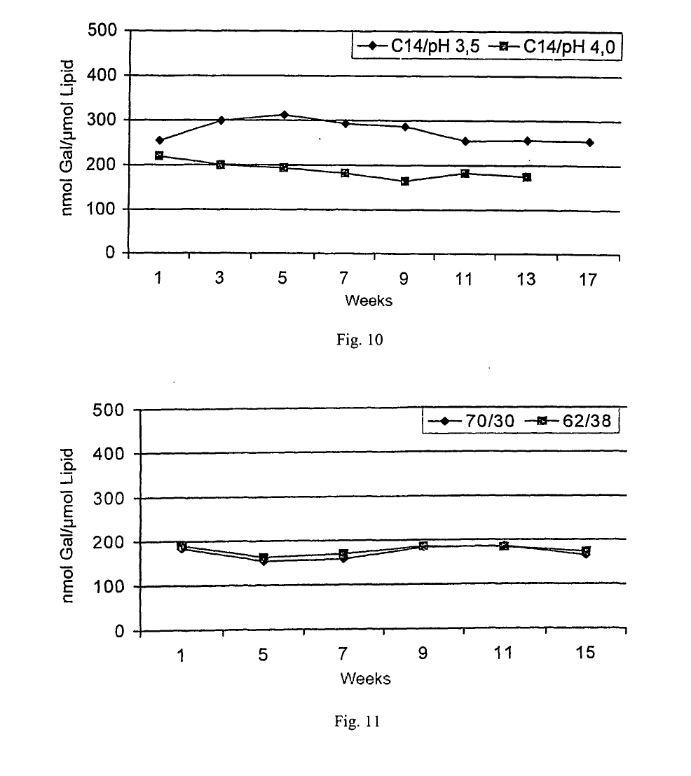

Liposome preparation, loading and analysis were carried out as in Example 1. Stability tests of liposome samples with DPPC:cholesterol molar lipid ratio=55:45 were continued. The results are given in FIGS. 9A and 9B.

FIG. 9A shows stability data of the first liposome sample successfully loaded with galantamine by the active method (three-step process). The liposomes were prepared in the presence of 0.3 mol citric acid (pH 3.5-4.5). After completed vesicle formation, galantamine was added and the pH of the solution outside of the liposomes was raised to 7.5 at the same time. The resulting pH gradient between the inside and outside of the liposomes led to the uptake of galantamine into the liposomes, as a function of the H⁺ ion concentration within the liposomes.

FIG. 9B shows stability data for liposome samples of similar composition, but where the vesicle formation and galantamine loading took place using the crossflow technique in a one-step process. The data clearly show that the pH gradient and thus the content of liposomally enclosed galantamine remained constant for a period of more than half a year.

To determine the best liposome formulation with regard to membrane flexibility and the related skin penetration properties, various liposome suspensions with different lipid compositions were prepared and tested. Phospholipids were used first of all, optionally in combination with cholesterol. However, it is within the scope of the invention to replace phospholipids with other lipids or to supplement them, for example, with glycolipids, cerebrocides, sulfatides or galactosides. Typical representatives of lipids that may be used are, for example, phosphatidylethanolamine, phosphatidylcholine, phosphatidylserine, phosphatidylinositol, phosphatidylglycerol, cardiolipin, sphingomyelins, plasmalogens, glyceroglycolipids, ceramides, glycosphingolipids, neutral glycosphingolipids.

One possibility for improving membrane fluidity, which is important for transdermal applications, is to reduce the phase transition of the liposomal bilayer, which is principally determined by the length of the acyl chains of the phospholipids, the amount of cholesterol and the saturation of the phospholipids. For this reason, DPPC, a phospholipid with acyl chain length of 16 carbon atoms, was replaced by DMPC (chain length 14 carbon atoms), which has a melting point TM of about 45-31° C.

FIG. 10 shows inclusion rates of liposome suspensions that were prepared by means of pH gradients 3.5-7.5 and 4.0-7.5. After satisfactory inclusion rates were achieved (comparable to those obtained using DPPC), samples were taken and tested for stability (FIG. 10).

Another possibility for reducing membrane rigidity and increasing fluidity is to reduce the amount of cholesterol in the membrane. Starting from a DPPC:cholesterol ratio of 55:45 mol % (as described in the literature for liposome loading), the amount of cholesterol was successfully reduced to 38% and to 30%, with respect to the total lipid content. In FIG. 11, one can see that there is a slight decrease of galantamine loading by comparison with previous data for higher cholesterol contents. However, these liposomes showed improved skin penetration properties, as will be described below. FIG. 11 also shows that the loaded liposomes remain stable in a long-term experiment and do not lose galantamine.

Counter to the findings from earlier studies, cholesterol-free liposomes could also be stably prepared and successfully loaded with active agent such that, in accordance with the invention, the cholesterol content lies in a range from 0-50 mol % with respect to the total lipid content.

A third possibility of making liposomal membranes more flexible is to replace the completely saturated DPPC or DMPC lipids with hen's egg phosphatidylcholine (E-PC), a natural lipid mixture with unsaturated phospholipids. In addition to stability problems when using these natural lipids, it was additionally necessary to prepare the liposomes under a nitrogen atmosphere. In spite of that, these vesicles did not give good results with respect to vesicle size, and homogeneity or with respect to improvements in skin penetration properties as described in Examples 3-10.

Besides the citric acid/sodium carbonate technique for producing a pH gradient, the ammonium sulfate gradient method is also often used. With this method, liposomes are formed in an ammonium sulfate buffer (125 mmol). After vesicle formation, the ammonium sulfate solution outside of the liposomes is replaced by a 5% glucose solution by means of difiltration, through which small amphiphilic molecules can be loaded into the liposomes and become protonated there, while NH₃ escapes from the liposomes in the counterflow. This operation is a milder process than the citric acid/sodium carbonate process.

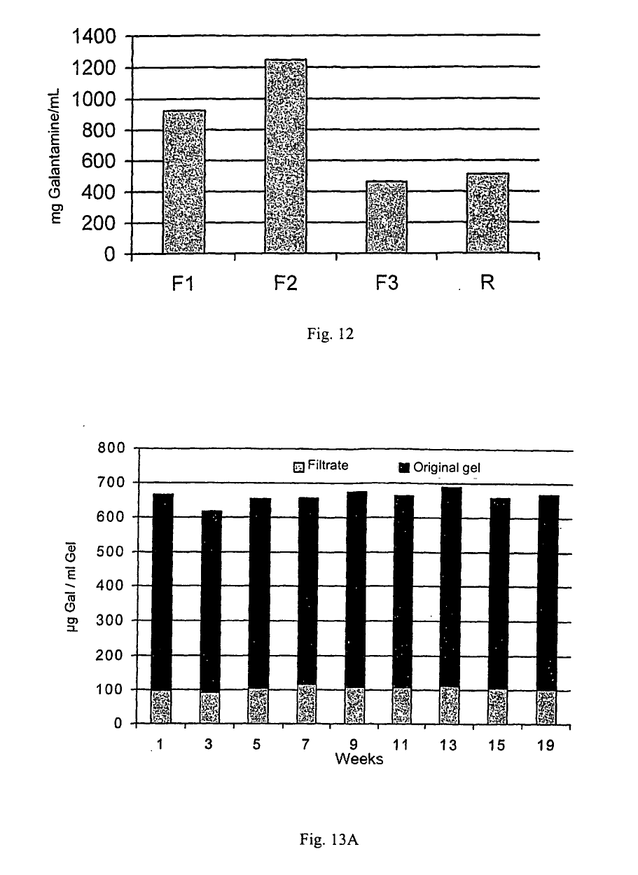

Nevertheless, these experiments did not give satisfactory results, at any rate when using galantamine as active agent to load the liposomes. The data in FIG. 12 show that the galantamine inclusion failed since the same amounts of active agent were found in the retentate and in the filtrate. This is why, for the inclusion of galantamine into liposomes, the citric acid/sodium carbonate method is to be preferred for external, active loading, though the use of alternative, well-known functional equivalent acid/base systems to form the desired pH gradient are also within the scope of this invention. Thus, citric acid could be replaced by another suitable pharmaceutically permissible acid, for example, a mineral acid like phosphoric acid, or preferably by an organic acid, especially one from the group of the edible organic acids like malic acid, fumaric acid, tartaric acid, optionally even ascorbic acid. In the same way, sodium carbonate could be replaced by another base, especially by another alkali or alkaline earth carbonate or bicarbonate.

“Functionally equivalent” in this connection is understood to mean the ability to be able to perform a pH gradient across the lipid bilayer of the liposomes and in doing so not to destroy the membrane integrity, so that enclosed, especially protonated active agents remain in the liposomes stably, in the sense of the stability criteria disclosed herein.

Preparation and Stability Testing of a Galantamine Liposome Gel:

For the use of a liposomal galantamine composition as a topical therapeutic agent, the liposomes are preferably mixed into a hydrogel, which is easier to apply to the skin than a pure suspension. However, it is also within the scope of this invention to prepare other galenical formulations for the galantamine liposomes and to apply them topically, especially formulations in the form of solutions, lotions, emulsions, tinctures, sprays, ointments, creams or optionally in the form of impregnated textile fabric or bandage materials. Other possibilities are familiar to specialists in the field, including the pharmaceutically acceptable auxiliary agents and additives that are needed to prepare the various galenical formulations.

For example, Carbopol 981NF, a hydrogel which can be used in very low concentrations, proved itself in earlier experiments. It is approved for pharmaceutical use, relatively cheap, and available in large quantities.

The following liposome gels were prepared for the penetration studies with the Franz diffusion cell:
DPPC:cholesterol=55:45/pH 3.5
DPPC:cholesterol=62:38/pH 3.5
DPPC:cholesterol=70:30/pH 3.5
DMPC:cholesterol=55:45/pH 3.5
E-PC:cholesterol=63:38/pH 3.5
E-PC:cholesterol=70:30/pH 3.5

For this purpose, the vesicle suspensions were concentrated by ultrafiltration and then mixed with a prepared sterile gel base while stirring. In this method the liposomal galantamine concentrations can be varied either via the ultrafiltration or via the initial concentration of the gel base, which is diluted with the vesicle suspension to a carbopol concentration of 0.5%.

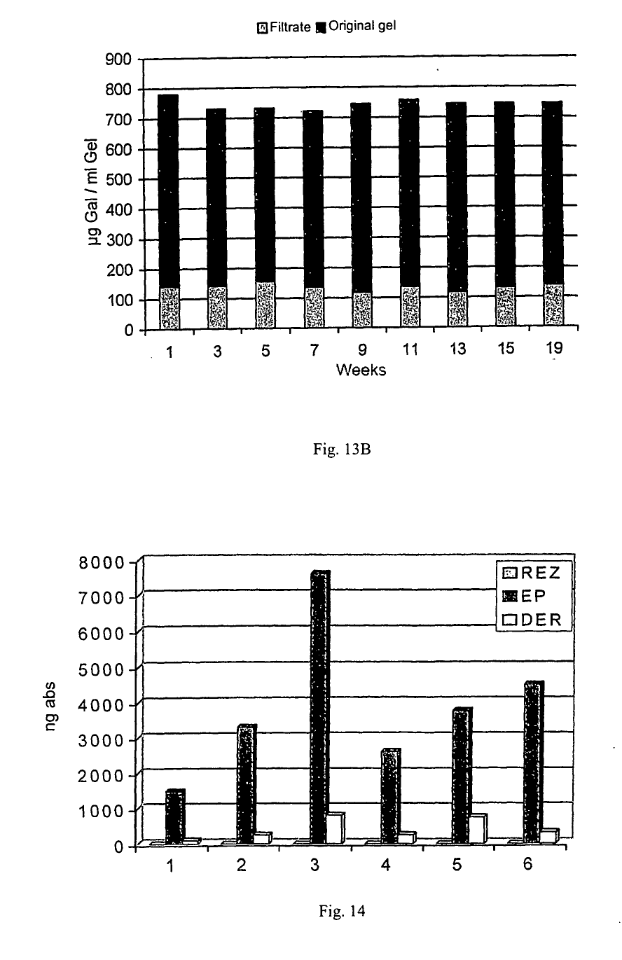

Standard testing was carried out for any loss of active substance that could have been caused by membrane damage during the galenical production process and/or by the long storage time. For this purpose, the gel was diluted with buffer and filtered. If there was membrane damage, larger amounts of released galantamine would be detectable in the filtrate. As can be seen from FIGS. 13A and 13B, significant amounts of active agent are not released either during the galenical formulation or during the subsequent storage at 4° C. over 19 weeks. Rather, the active substance remains in the liposomes even after the galenical formulation with the carbopol hydrogel and it shows the same penetration profile in the skin test, which proves that both the liposomes and the pH gradient in the gel matrix remained intact.

For comparison of the skin penetration properties, free galantamine was also mixed with hydrogel in the exact same manner and tested. The results are presented in Examples 3-10.

Alternative Concepts:

Besides liposomes, microemulsions have also increasingly attracted attention in recent years for topical applications of certain active substances. Microemulsions are dispersions of two mutually immiscible components, stabilized by a third amphoteric component. However, because of the presence of surface-active substances like emulsifiers and coemulsifiers, microemulsions can damage the skin in a manner similar to transdermal patches.

According to the above literature studies, a number of microemulsions (Table 1) were prepared with galantamine as active substance and then subjected to penetration tests with the Franz diffusion cell (results, see Examples 3-10).

TABLE 1
Microemulsions

| Lecithin ME | W/O ME | O/W ME |
|---|---|---|
| 10 mL IPM | 7.2 mL IPM | 5.5 g H₂O |
| 1.9 g SPC | 0.2 g Chol | 2.5 g IPM |
| 135 μL H₂O | 0.5 g H₂O | 2 g Tween/Span 20 |
| 10 mg Gal | 2 g Tween/Span 20 | 11 mg Gal-HBr |
| | 10 mg Gal | 5 mg Gal |

ME = microemulsion;
W/O = water-in-oil;
O/W = oil-in-water

In all the experiments described here, it turned out that only a reduction of the cholesterol content of the liposome membrane brought an improvement of the transdermal penetration properties. The prepared liposome suspensions and liposomal gel preparations are stable for more than half a year and do not show any changes in the product properties.

### EXAMPLES 3-10

### Skin Penetration Tests of Various Lipid-Based Galantamine Preparations Franz Diffusion Cell

The diffusion cell came from PermeGear, USA. The equipment consisted of three diffusion cells, each with a water-filled double jacket mounted on an agitator bracket and connected to a water bath for temperature control. The cells themselves had a receptor volume of 8 mL each, a skin holder with a surface area of 0.78 cm² and a donor chamber with 2 mL volume.

Skin Integrity Test:

Pigskin was used for the tests in the Franz diffusion cell. In order to ensure an intact skin surface, each skin piece was tested before and after the experiment. After affixing the skin on the skin holder of the diffusion cell, 2 mL buffer was applied to the skin and it was heated to 32±1° C. After 30 minutes, the electrical conductivity, a measure of the resistance of the skin and quality of the skin, was measured. The measurement value is dependent on the origin of the skin, its thickness, the buffer system that is used and the equipment used for the measurement. Based on several preliminary experiments, a limit value of ≦1 mS/cm² for the electrical conductivity of the intact skin before application of a sample was established. Skin samples that did not meet this requirement were not used for the penetration experiments.

Penetration Buffer:

Since the liposomes in accordance with the invention were prepared in 0.3M citric acid buffer, which had been adjusted to pH 7.5 with 1M sodium carbonate solution, the same buffer was also used for all of the penetration experiments.

Sample Handling after the Penetration Experiment:

After the end of each experiment, the excess galantamine sample was removed by washing the surface with buffer. Then the electrical conductivity was measured again and the skin sample was removed from the holder. For purposes of separating the epidermis (top layer) from the dermis (true skin), the skin sample was placed on an electric hotplate and heated for 30 seconds at 60° C. After this heat treatment, the epidermis can be lifted quite simply by means of a pincette. Immediately after that, the epidermis and dermis were separately placed in plastic test tubes and frozen at −20° C. For galantamine extraction, 300 μL buffer was added to the frozen sample in the presence of liquid nitrogen, and the deep frozen sample was then pulverized in a cryomill. The powder was immediately transferred to a centrifuge tube and centrifuged at 4° C., after which the clear supernatant was transferred to a clean test tube and refrozen at −20° C. until the start of the analysis.

Quantification of the Galantamine Supernatant:

A modified, more sensitive and faster rp-HPLC method was set up for the determination of small amounts of galantamine:

| | |
|---|---|
| HPLC: | Agilent 1100 |
| Column: | Thermo Hypersil Keystone |
| | 150 × 4.6 mm, 5 μm, 190 A |
| Gradient: | linear gradient |
| | Solvent A: H₂O/0.1% TFA |
| | Solvent B: ACN/0.1% TFA |
| Detection: | DAD, 230 nm |
| Quantification range: | 30-1000 ng/mL |
| Injection volume: | 100 μL |

Test Material:

Several samples with different galantamine formulations were tested. Changes in input volume and penetration design were carried out and are summarized in the results.

Material:
Liposomal galantamine suspensions:
C16/3.5/(55/45)
C14/3.5/(55/45)
Liposomal galantamine gels:
C16/3.5/(55/45)
C16/3.5/(62/38)
C16/3.5/(70/30)
C14/3.5/(55/45)
E-PC/3.5/(62/38)
E-PC/3.5/(70/30)
Free galantamine gels:
2-20 mg/g gel
Galantamine microemulsions:
Lecithin ME (1 mg/mL)
Water/oil (W/O) ME (1 mg/mL)
Oil/water (O/W) ME (1.6 mg/mL)

The various liposomal galantamine formulations were prepared with the crossflow injection technique. The material was tested both in suspension and in a carbopol (981NF) gel matrix. Variations in the membrane composition of the liposomes were obtained through the use of different lipids and different cholesterol contents (see Examples 1 and 2).

The following penetration studies were conducted with different sample volumes and single and multiple sample inputs, where samples for galantamine determination were taken at timed intervals. The results shown in FIGS. 14 to 21 are each based on a three-fold penetration experiment. The diagrams show the results in ng galantamine absolute per analyzed sample. The results are divided into values that were determined from the receptor liquid REZ (liquid that penetrated the skin and was collected in the receptor chamber) and those that were determined from the epidermis (EP) or dermis (DER).

### EXAMPLE 3

Example 3 compares the results of different DPPC (C16) liposomes having different cholesterol contents with those of DMPC (C14) liposomes and E-PC liposomes. A sample volume of 50 μL was input once and left to penetrate for a period of 4 hours. All of the tested preparations had comparable amounts of enclosed galantamine and were suspended in 0.5% carbopol 981NF. The most effective formulation in this experiment was the sample with the C16/70/30 (acyl chain length/mol % phospholipids/mol % cholesterol) lipid composition. In this gel, the cholesterol concentration in the liposomes was 30 mol %. The other two preparations had considerably higher cholesterol contents (38 and 45 wt %), and were thus also considerably more rigid. The data determined from this experiment confirmed the theory that more highly fluid liposomes, i.e., less rigid membranes, penetrate more efficiently (FIG. 14).

In FIG. 14:
1=C16/55/45;
2=C16/62/38;
3=C16/70/30;
4=C14/55/45;
5=E-PC/62/38;
6=E-PC/70/30.

### EXAMPLE 4

In Example 4, the penetration properties of gel samples were tested after repeated application to the skin. Two different gels with different lipid compositions and the same cholesterol content were compared. The samples were each applied three times in amounts of 50 μL, specifically at intervals of 4 hours. The excess material in each case was removed before applying the next sample.

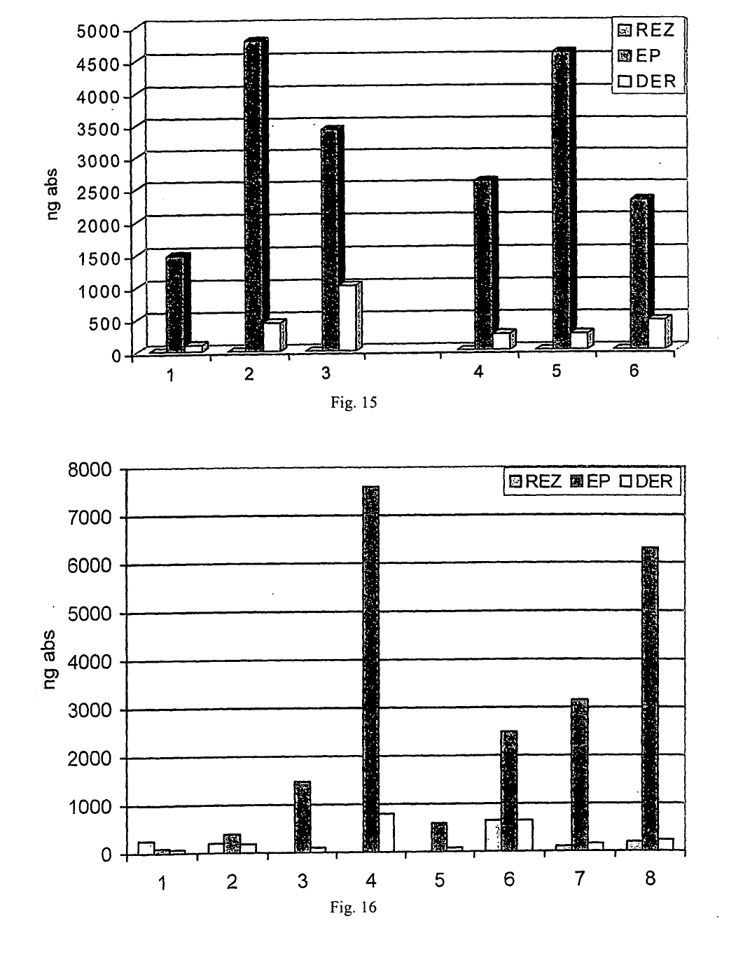

As can be seen from the diagram (FIG. 15), at the end, an increase of the galantamine concentration could be achieved, but the penetration into the epidermis appears to be hindered or slowed in some way with application of the material three times. Possibly this is a kind of saturation caused by the small surface area of the clamped skin piece in combination with the high application doses. Comparable results were achieved for both samples, so that an effect on the part of the lipid composition was not obviously detectable (FIG. 15).

In FIG. 15:

(chain length/phospholipids/cholesterol/sample amount total)
1=C16/55/45/50 μL;
2=C16/55/45/100 μL;
3=C16/55/45/150 μL;
4=C14/55/45/50 μL;
5=C14/55/45/100 μL;
6=C14/55/45/150 μL.

### EXAMPLE 5

In Example 5, gels containing C16 liposomes with high and low cholesterol contents were compared after a single application. Sample volumes of 150 μL and 50 μL were left to penetrate for 4 and 10 hours. The highest amount of galantamine in the skin was again found when using liposomes with low cholesterol content. Here, too, low dosages appear to be more advantageous. After 4 and 10 hours, high galantamine quantities were found in the epidermis when 50 μL was used. These results confirm the values that were achieved in Examples 3 and 4, i.e., the administration of liposomes with low cholesterol content with simultaneously low application amounts overall could be a favorable strategy in the topical use of the preparation in accordance with the invention in prophylactic or therapeutic use (FIG. 16).

In FIG. 16:

(chain length/phospholipids/cholesterol/sample amount/penetration time)
1=C16/55/45/150 μL/4 h;
2=C16/70/30/150 μL/4 h;
3=C16/55/45/50 μL/4 h;
4=C16/70/30/50 μL/4 h;
5=C16/55/45/150 μL/10 h;
6=C16/70/30/150 μL/10 h;
7=C16/55/45/50 μL/10 h;
8=C16/70/30/50 μL/10 h.

### EXAMPLE 6

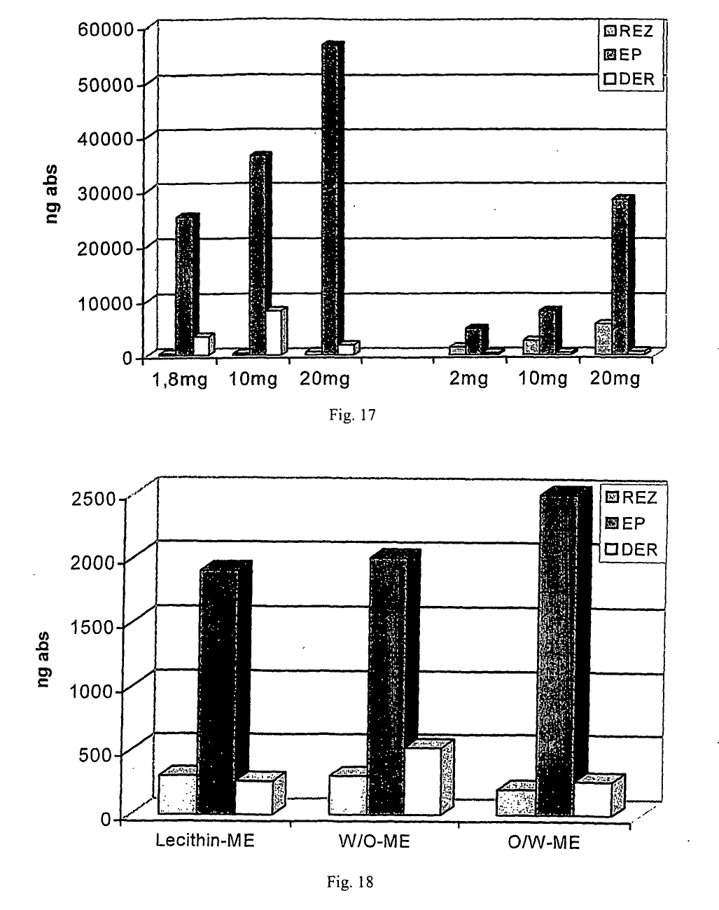

In Example 6, the effect of the gel concentration was tested in connection with three different concentrations on free galantamine suspended in the gel. 50 μL of each formulation was applied one time and the penetration experiment was carried out for 4 and 10 hours (FIG. 17). The first three bars in FIG. 17 represent the results with 1% carbopol 981NF after 4 hours, while the next three represent the results with 0.5% carbopol 981NF after 10 hours. The results after 4 hours show that free galantamine diffuses into the skin tissue relatively rapidly. To be sure, as can be seen from the 10-hour values, the free agent was also found in a high concentration in the receptor liquid, so that only a negligible depot effect should be expected (FIG. 17).

### EXAMPLE 7

In Example 7, different application strategies were tested. It is known from the literature that microemulsions can be useful tools as carrier systems for administration of small amphiphilic molecules. To test this concept, various microemulsions (lecithin; water-in-oil; oil-in-water) were prepared, each with 1 mg galantamine per mL (see Examples 1 and 2).

Sample volumes of 50 μL were each applied one time and the penetration was carried out over a period of 4 hours. As can be seen from the diagram (FIG. 18), the absolute amounts of galantamine in the skin were lower than when using the hydrogel with liposomes. Moreover, the active agent was uniformly distributed in the dermis (top layer) and receptor fluid, which indicates that rapid penetration with, at best, marginal storage in the skin tissue took place (FIG. 18). These results agree with those of other authors and point to a penetration mechanism that is similar to the use of transdermal galantamine patches.

### EXAMPLE 8

In Example 8, the results with free galantamine in hydrogel and microemulsions were compared to those of the preferred liposome composition (C16 phospholipids with low cholesterol fraction). In all of the experiments, comparable galantamine concentrations were tested under similar conditions.

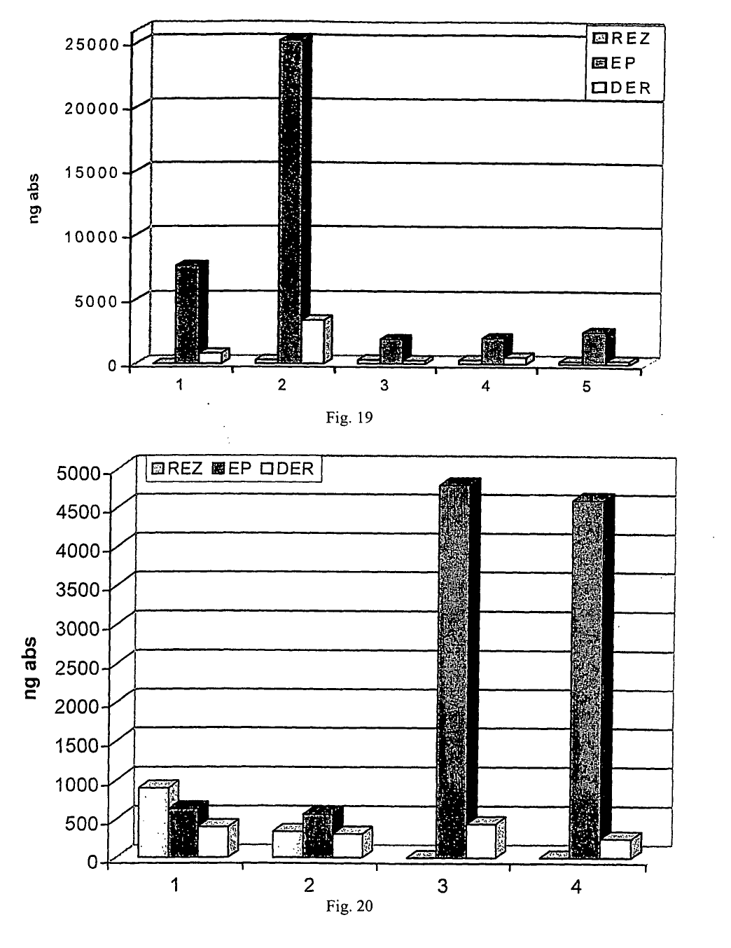

As can be seen from the diagram (FIG. 19), considerable amounts of galantamine were found in the skin when using the hydrogel formulation with free galantamine. Acceptable values were also obtained with the liposome formulation, whereas less acceptable results were obtained with the microemulsions. However, as already explained in the preceding examples, the goal of a new topical active agent formulation is not firstly a rapid skin penetration, but rather the ability to form an active agent depot in the skin from which the active agent can be slowly released. Such a depot could, on the one hand, reduce the frequency of application and, on the other hand, bring about a more uniform controlled release of the active agent, which would improve the therapeutic effect. This is achieved when the active agent becomes stored in the upper layers of the skin and not in deeper regions, as in the case with the liposomal formulations in accordance with the invention.

In FIG. 19:
1=liposomes (C16/70/30);
2=1.8 mg galantamine, free in hydrogel;
3=1.8 mg free galantamine in lecithin microemulsion;
4=1.8 mg free galantamine W/O ME; and

5=1.8 mg free galantamine in O/W ME.

### EXAMPLE 9

In Example 9, liposomal formulations in suspension and in hydrogel were compared to each other. The administration of an excess of 1 mL liposomal suspension over a period of at least 24 hours led only to low penetration effectiveness (FIG. 20). Therefore, it appears to be confirmed that a suitable gel matrix not only stabilizes the liposomes and makes the application easier and more agreeable, but also brings the liposomes closer to the skin and thus increases the penetration effect.

In FIG. 20:

(chain length/phospholipids/cholesterol/sample amount/penetration time/type)
1=C16/55/45/1 mL/24 h/suspension;
2=C14/55/45/1 mL/24 h/suspension;
3=C16/55/45/100 μL/8 h/gel;
4=C14/55/45/100 μL/8 h/gel.

### EXAMPLE 10

In general, the in vitro test using the Franz diffusion cell appears to be a useful method for penetration studies with various liposomal formulations, even though certain limits of the applicability of the method, particularly when using liposomal gel formulations, came to light. Based on earlier experience with various liposome gels in vivo, it was also expected for the in vitro penetration tests described here that each gel can penetrate into the skin completely without leaving an excess on the skin surface. Nevertheless, we were unable to administer the gel with the same success in the experiments with the Franz diffusion cell. In all of the experiments, a considerable excess of sample material remained on the skin surface. This disadvantage presumably had a negative effect on the penetration efficiency. Therefore, it can be expected that higher amounts of liposomal galantamine will penetrate into the skin in vivo.

Still, even in the in vitro experiments with the various liposomal formulations described here, up to 1.8×10¹⁶ active agent molecules were transported into the skin (epidermis) per 0.78 cm² skin surface.

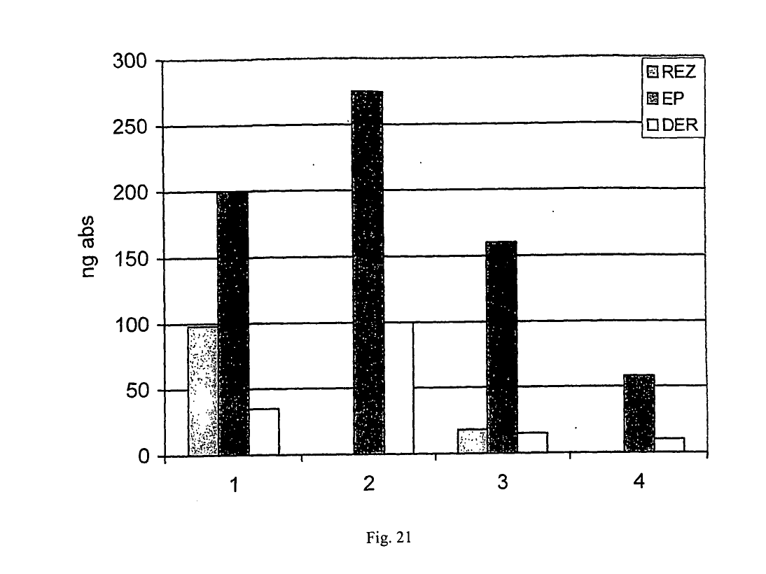

In order to see if possibly the removal of the skin using the dermatome or the pretreatment of the skin could be responsible for the observed application problems, various skin samples were tested (FIG. 21). However, a clear effect could not be established from the results. Therefore, one should assume that the skin itself does not have a significant negative effect on the achieved results.

In FIG. 21:
1=skin, untreated; liposomes; 70/30 (phospholipids/cholesterol);
2=skin cleaned with ethanol; liposomes: 70/30;
3=skin treated with oily gauze; liposomes: 55/45;
4=skin cleaned with ethanol and treated with oily gauze; liposomes: 55/45.

At any rate, one can say in conclusion that liposomal galantamine formulations in a hydrophilic gel are an advantageous presentation form when the site of treatment lies in the dermal tissue (true skin), even if the active agent is applied to the skin only twice a day. Through the mild, non-invasive and non-skin-irritant use of the liposomally enclosed active agent in accordance with the invention, a considerable depot effect in the epidermis and a slow uniform release of the active agent into the underlying dermal tissue can be achieved.

Abbreviations:

ACN acetonitrile

DAD diode array detector

DER dermis (true skin)

DMPC dimyristoylphosphatidylcholine

DPPC dipalmitoylphosphatidylcholine

E-PC natural phosphatidylcholine from eggs

E-PG natural phosphatidylglycerol from eggs

EP epidermis (top skin)

IPM isopropyl myristate

NGF nerve growth factor

PBS phosphate buffered saline

REZ receptor vessel (receptor chamber)

rp-HPLC reversed phase high performance liquid chromatography

TFA trifluoroacetic acid

1. A pharmaceutical composition for topical, transdermal administration comprising liposomes, the liposomes having an acid, aqueous environment in their interior and containing therein at least one cholinesterase inhibitor.

2. A composition of claim 1, wherein the pH of the aqueous environment within the liposomes is in the range of 2.5 to 5.5.

3. A composition of claim 1, wherein the aqueous environment within the liposomes comprises an organic acid.

4. A composition of claim 1, wherein an environment with neutral or alkaline pH value is present outside the liposomes.

5. A composition of claim 1, wherein the liposomes are unilamellar and have a lipid bilayer.

6. A composition of claim 1, wherein the liposomes comprise phospholipids with an acyl chain length of at least 14 carbon atoms.

7. A composition of claim 1, wherein the liposomes comprise cholesterol in an amount of 0 to 50 mol % of the total lipids.

8. A composition of claim 1, wherein the liposomes have an average size in the range of 150 to 500 nm.

9. A composition of claim 1, wherein the liposomes contain the active agent in a concentration of at least 100 nmol per μmol of lipid.

10. A composition of claim 1, wherein the composition is in the form of a suspension, lotion, emulsion, tincture, spray, gel, cream or ointment.

11. A method for preparing a pharmaceutical composition comprising an active agent enclosed in liposomes, the method comprising injecting an ethanol liquid phase into an acid aqueous phase and thereby spontaneously generating liposomes with an acid, aqueous environment in their interior, after which the aqueous phase is neutralized or made alkaline, so that a pH gradient is formed between the inside and outside of the liposomes.

12. A method of claim 11, wherein the neutralization or alkalinization is carried out immediately after completion of liposome formation.

13. A method of claim 12, wherein the aqueous phase before the neutralization or alkalinization has a pH value of 2.5 to 5.5 and afterward has a pH value of 7 to 8.

14. A method of claim 11, wherein the acid aqueous phase comprises an organic acid and the neutralization or alkalinization is undertaken by diluting the aqueous phase with an alkaline buffer.

15. A method of claim 11, wherein the lipid phase comprises phospholipids with an acyl chain length of at least 14 carbon atoms.

16. A method of claim 11, wherein the liposomes comprise cholesterol in an amount 0 to 50 mol % of the total lipids.

17. (canceled)

18. A method of claim 11, wherein the pharmaceutical composition with the liposomes loaded with active agent is prepared in the form of a suspension, lotion, emulsion, tincture, spray, gel, cream or ointment.

19. A method of treatment comprising topically applying to the skin of a patient a pharmaceutical composition of claim 1.

20. A method of claim 19 wherein the treatment is for prophylaxis and/or therapy of dermal neuropathic pain or neuropathy-related loss of dermal sensory function.

21. A method of claim 20, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers and derivatives of at least one of these compounds.

22. A method of claim 19, wherein the method reduces or avoids undesirable systemic side effects as compared to the at least one cholinesterase inhibitor not enclosed in liposomes.

23. A composition of claim 1, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers and derivatives of at least one of these compounds.

24. A composition of claim 1, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers of at least one of these compounds.

25. A composition of claim 1, wherein the pH of the aqueous environment within the liposomes is in the range of 3.5 to 4.5.

26. A composition of claim 1, wherein the aqueous environment within the liposomes comprises citric acid.

27. A composition of claim 4, wherein the environment outside the liposomes has a pH from 7 to 8.

28. A composition of claim 4, wherein the environment outside the liposomes has a pH of 7.5.

29. A composition of claim 1, wherein the liposomes comprise phospholipids with an acyl chain length of at least 16 carbon atoms.

30. A composition of claim 1, wherein the liposomes comprise cholesterol in an amount of 30 to 45 mol % of the total lipids.

31. A composition of claim 1, wherein the liposomes contain the active agent in a concentration of at 150 to 400 nmol per μmol of lipid.

32. A composition of claim 10, wherein the composition is in sterile form.

33. A method of claim 11, wherein the active agent is present in the acid aqueous phase and is taken up into the liposomes in the course of the spontaneous liposome formation.

34. A method of claim 11, wherein the active agent is not added to the aqueous phase until liposome formation is complete and then migrates into the liposomes along the pH gradient.

35. A method of claim 12, wherein the aqueous phase before the neutralization or alkalinization has a pH value of 3.5 to 4.5 and afterward has a pH value of 7.5.

36. A method of claim 14, wherein the organic acid is citric acid and the alkaline buffer is sodium carbonate.

37. A method of claim 11, wherein the lipid phase comprises phospholipids with an acyl chain length of at least 16 carbon atoms.

38. A method of claim 11, wherein the liposomes comprise cholesterol in an amount of 30 to 45 mol % of the total lipids.

39. A method of claim 33, wherein at least one cholinesterase inhibitor is present as active agent in the acid aqueous phase.

40. A method of claim 34, wherein at least one cholinesterase inhibitor is the active agent and is added to the aqueous phase after completion of liposome formation.

41. A method of claim 39, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers and derivatives of at least one of these compounds.

42. A method of claim 39, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers of at least one of these compounds.

43. A method of claim 40, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers and derivatives of at least one of these compounds.

44. A method of claim 40, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers of at least one of these compounds.

45. A method of claim 18, wherein the pharmaceutical composition is in sterile form.

46. A method of claim 20, wherein the at least one cholinesterase inhibitor is selected from the group consisting of donepezil, rivastigmine, galantamine, physostigmine, heptylphysostigmine, phenserine, tolserine, cymserine, thiatolserine, thiacymserine, neostigmine, huperzine, tacrine, metrifonate and dichlorvos, and enantiomers of at least one of these compounds.
